(19) United States
(12) Patent Application Publication
(10) Pub. No.: US 2020/0155348 A1
(43) Pub. Date: May 21, 2020

(54) CAPSULORHEXIS APPARATUS

(71) Applicant: TI INC., Gyeonggi-do (KR)

(72) Inventors: Hong Jai LEE, Seoul (KR); Sung Hyuk MOON, Busan (KR); Jae Wook YANG, Busan (KR); Seung Jai LEE, Gyeonggi-do (KR); Shigenori TOYAMA, Seoul (KR)

(21) Appl. No.: 16/636,335

(22) PCT Filed: Aug. 16, 2017
(86) PCT No.: PCT/KR2017/008908

§ 371 Date: Feb. 3, 2020

(30) Foreign Application Priority Data
Aug. 4, 2017 (KR) 10-2017-0099049

(51) Int. Cl. A61F 9/007

(52) U.S. Cl. CPC A61F 9/00754

(57) ABSTRACT

The present invention provides a capsulorhexis apparatus for incising the anterior capsule covering the crystalline lens by being inserted into an incision site of the cornea. The capsulorhexis apparatus comprises: an anterior capsule incision part which has a closed curve-shape, is inserted in an incision site of an incised cornea so as to incise one portion of the anterior capsule positioned at the lower part of the cornea, and generates heat by applying a current in order to incise the anterior capsule; a body part in which the anterior capsule incision part slidingly moves so that the anterior capsule incision part is exposed to the outside when same is incising one portion of the anterior capsule, and is inserted inside the body part when same is not incising the anterior capsule; and a button part which is formed on one surface of the body part and slidingly moves the anterior capsule incision part inside the body part. Accordingly, the front surface of the anterior capsule covering the crystalline lens may be precisely incised in a circular shape, and thus revision surgery as a result of a failed surgery may be prevented, and the surgical procedure may be safely and conveniently conducted.

### TECHNICAL FIELD

The present invention relates to a capsulorhexis apparatus, and more particularly, to a capsulorhexis apparatus for clearly and precisely incising a front surface of an anterior capsule covering the crystalline lens of an eyeball in a circular shape.

### BACKGROUND ART

Referring to the background art and FIG. 1 of Korean Patent Application Publication No. 2010-0016724, an eye (or an eyeball) 1 included in a body is an organ which secures a clear view by sensing the intensity and wavelength of light. The eye 1 includes a cornea 20 formed to surround an outer portion of a sclera 10, a crystalline lens 30 which is colorless and transparent and serves a role like a lens of a camera, an iris 40 which decides the color of the pupil due to the presence of a pigment and serves as an aperture for adjusting the amount of light incident on the eye, a retina (not shown) which corresponds to film of the camera and includes a transparent nerve system, and the like.

Meanwhile, the crystalline lens 30 included in the eye as described above corresponds to the lens of the camera. When the lens of the camera gets dirty, sharpness of a picture is degraded. Likewise, when the crystalline lens 30 is clouded, light is not passed through the eye well, and thus objects look blurred. As such, the crystalline lens 30 that is clouded due to various reasons is referred to as a cataract.

When cataracts develop, it is necessary to prevent visual power from being degraded or lost through prompt treatment. As a treatment for cataracts, a method of incising a lens capsule 50 covering the crystalline lens 30, fragmenting the crystalline lens 30 in the lens capsule 50 with supersonic waves, removing the fragmented crystalline lens 30 with supersonic waves or the like, and inserting an artificial crystalline lens (not shown) instead of the removed crystalline lens 30 is widely used.

In other words, the sclera 10 or the cornea 20 is incised to a length of about 2 mm to 3 mm by using a diamond knife or the like, and an incision tool (not shown) which is formed by bending the tip of a needle or the like is inserted into an incision site 22 to rake out the front surface of the lens capsule 50 in a predetermined shape. The crystalline lens 30 exposed accordingly is fragmented with supersonic waves. Then, the fragmented crystalline lens 30 is aspirated and externally discharged, and in its place, instead, the artificial crystalline lens is inserted and fixed therein.

However, as a conventional incision tool, the operator should insert a bent needle, a pair of forceps, or the like into the incision site 22 and then rake out the lens capsule 50 several times to incise the front surface of the lens capsule 50.

Therefore, with such an incision tool, it is difficult to incise the lens capsule 50 in an appropriate size and shape (particularly in a circular shape) clearly and precisely. In some cases, problems, such as a radial rupture of an anterior capsule, occur, and an artificial crystalline lens (not shown) is not appropriately inserted. Consequently, endophthalmitis may occur, or a revision surgery such as changing artificial crystalline lenses may be required.

Also, it is necessary to move the incision tool several times very carefully in order to incise the front surface of the lens capsule 50. Therefore, the surgical procedure is difficult and, accordingly, takes a long time.

### DISCLOSURE

### Technical Problem

The present invention is directed to providing a capsulorhexis apparatus which reduces a procedure time by allowing an anterior capsule covering the crystalline lens of an eyeball to be precisely incised in a circular shape, allows an anterior capsule incision to be made with low voltage and low variability by allowing a current to flow through an incision tool itself, and thus allows a surgical procedure to be safely and conveniently conducted.

### Technical Solution

One aspect of the present invention provides a capsulorhexis apparatus that is inserted into an incision site of a cornea and incises an anterior capsule covering a crystalline lens, the capsulorhexis apparatus including an anterior capsule incision unit which has a closed curve shape and configured to be inserted into an incision site of an incised cornea and incise a part of the anterior capsule positioned under the cornea and generate heat by applying a current in order to incise the anterior capsule, a body unit in which the anterior capsule incision unit slidingly moves so that the anterior capsule incision unit is exposed to an outside of the body unit when incising a part of the anterior capsule and so that the anterior capsule incision unit is inserted into the body unit when not incising the anterior capsule, and a button unit which is formed on a surface of the body unit and slidingly moves the anterior capsule incision unit in the body unit.

The anterior capsule incision unit may include a circular incision tool configured to incise the part of the anterior capsule in a circular shape, a moving member having one end coupled to the circular incision tool and another end coupled to the button unit and configured to slidingly move the circular incision tool by manipulation of the button unit, and a current supply member configured to apply the current to the circular incision tool.

The circular incision tool may include a first wire which has elasticity and in which the current applied from the current supply member flows and a second wire which has elasticity and in which the current applied from the first wire flows and may further include a coated wire provided between the first wire and the second wire and having a circumferential surface coated with an insulating coating layer.

The first wire and the second wire may be interwoven in a rope shape and may have a metal material.

The anterior capsule incision unit may further include a current guide member configured to slidingly move and be inserted into the body unit or exposed to the outside of the body unit and configured to be positioned at a center of the circular incision tool and guide a current flow to the center of the circular incision tool when exposed to the outside of the body unit.

The anterior capsule incision unit may further include a first button unit formed to be spaced apart from the button unit on the surface of the body unit and configured to slidingly move the current guide member to be inserted into the body unit or exposed to the outside thereof.

The current guide member may include a moving bar having an end coupled to the first button unit and configured to move another end to the center of the circular incision tool by manipulation of the first button unit and a current guide piece provided at the other end of the moving bar and configured to guide a current flow in the circular incision tool. The current guide piece may be integrally provided at the other end of the moving bar.

The body unit may include a guide unit configured to cause one end of the anterior capsule incision unit to be inserted into the incision site of the cornea while slidingly moving in the guide unit and configured to guide the anterior capsule incision unit through the incision site of the cornea and a body which has an end coupled to the guide unit and in which a moving member slidingly moves. The button unit may be provided on a surface of the body.

The guide unit may have a silicone material, a moving hole, in which the anterior capsule incision unit slidingly moves, may be formed at a center of the guide unit, and an end of the guide unit coupled to the body may be obliquely coupled to the body.

The anterior capsule incision unit may include a first semicircular incision tool configured to incise the anterior capsule in a semicircular shape, a second semicircular incision tool having a shape corresponding to the first semicircular incision tool and configured to incise the anterior capsule in a semicircular shape, a first moving member having an end coupled to the first semicircular incision tool and another end coupled to the button unit and configured to slidingly move the first semicircular incision tool by manipulation of the button unit, a second moving member having an end coupled to the second semicircular incision tool and another end coupled to the button unit and configured to slidingly move the second semicircular incision tool by manipulation of the button unit, and a current supply member configured to apply the current to the first semicircular incision tool and the second semicircular incision tool.

The current may be applied from the current supply member to the first semicircular incision tool, and the current may be applied from the first semicircular incision tool to the second semicircular incision tool.

### Advantageous Effects

A capsulorhexis apparatus according to the present invention has the following effects.

First, it is possible to precisely incise the front surface of an anterior capsule covering a crystalline lens in a circular shape. Consequently, revision surgery as a result of a failed surgery may be prevented, and the surgical procedure may be safely and conveniently conducted.

Second, a circular incision tool of an anterior capsule incision unit for incising an anterior capsule is formed by interweaving first and second wires so that a current does not flow through a human body but flows only through the circular incision tool. Consequently, it is possible to make an incision with stable power.

Third, since a circular incision tool is formed by interweaving first and second wires, the first and second wires of the circular incision tool come into contact with an anterior capsule in alternate position. Consequently, it is possible to partially incise the anterior capsule in a circular shape.

### DESCRIPTION OF DRAWINGS

FIG. 1 is a schematic view illustrating an eyeball of a human body.

FIG. 2 is a schematic view illustrating a capsulorhexis apparatus according to an embodiment of the present invention.

FIG. 3 is a plan view of the capsulorhexis apparatus shown in FIG. 2.

FIG. 4 is a view illustrating a state in which an anterior capsule incision unit shown in FIG. 2 is inserted into a guide unit.

FIG. 5 is a view illustrating a capsulorhexis apparatus to which a modified anterior capsule incision unit is applied.

FIG. 6 is a schematic view illustrating a capsulorhexis apparatus to which another anterior capsule incision unit is applied.

FIG. 7 is a set of diagrams illustrating a process of incising an anterior capsule with a capsulorhexis apparatus according to the present invention.

### MODES OF THE INVENTION

Hereinafter, exemplary embodiments of the present invention will be described in detail with reference to the accompanying drawings. Terms or words used in this specification and the claims are not to be construed as common or dictionary meanings but are to be construed as meanings and concepts in accordance with the technical spirit of the present invention based on a principle that the inventor may define terms appropriately so as to best explain his or her own invention.

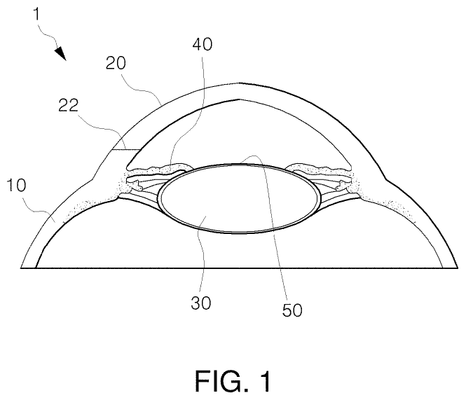

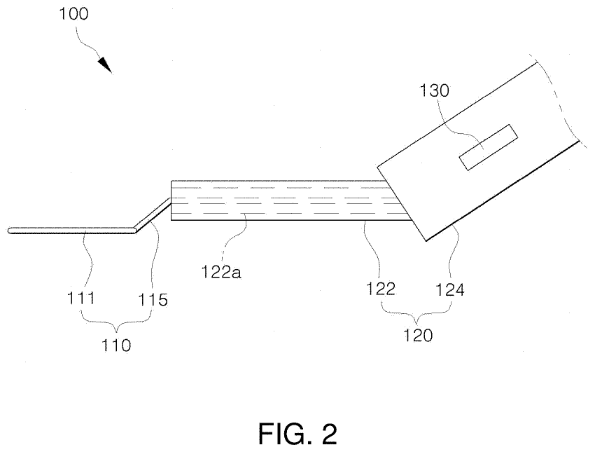

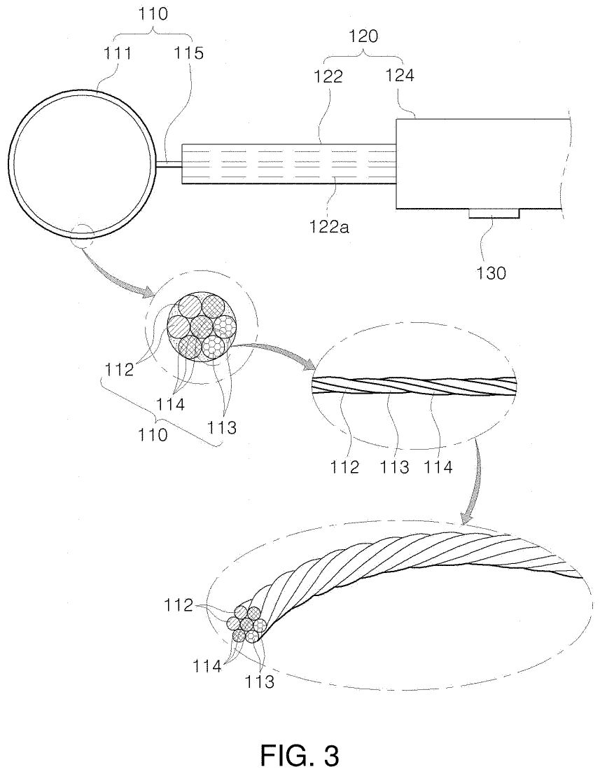

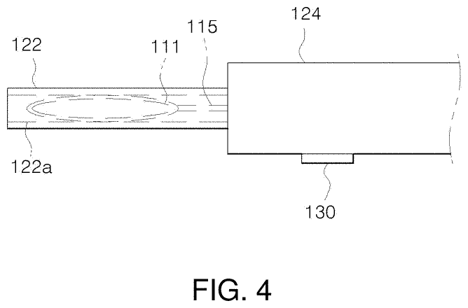

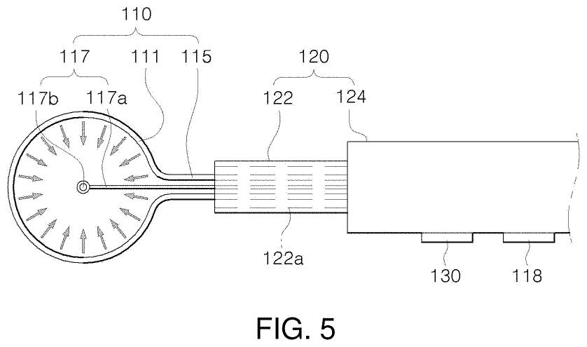

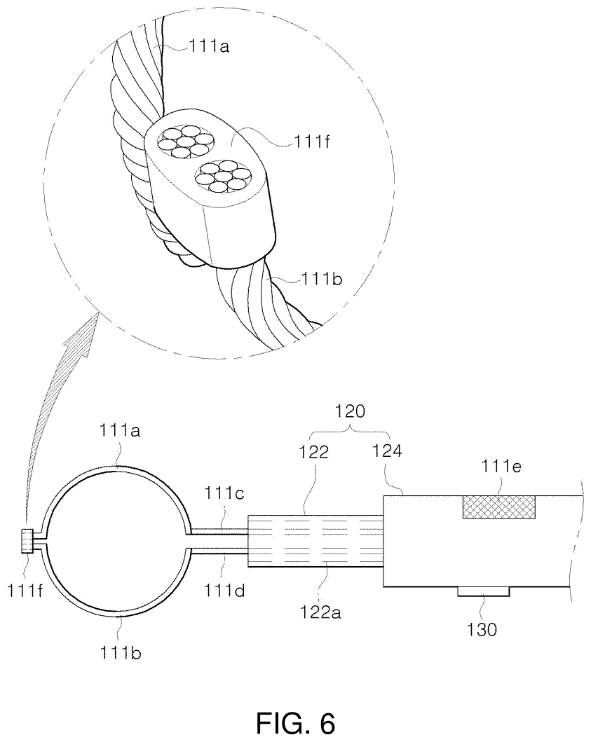

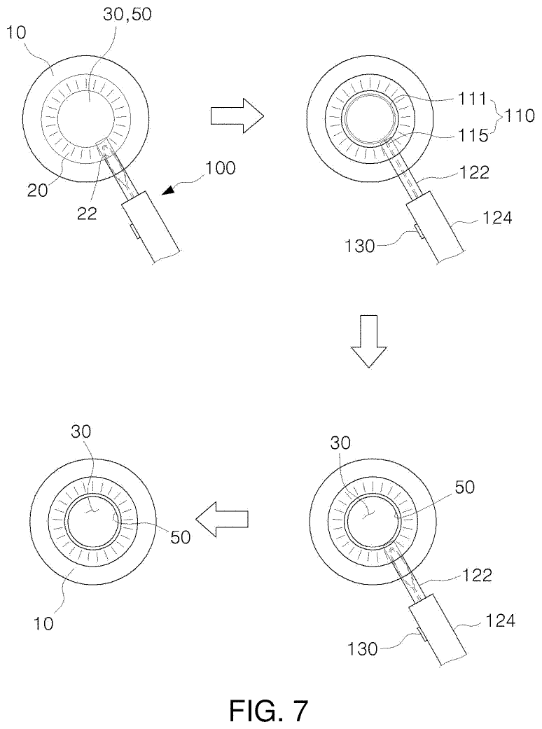

FIG. 1 is a schematic view illustrating an eyeball of a human body, FIG. 2 is a schematic view illustrating a capsulorhexis apparatus according to an embodiment of the present invention, FIG. 3 is a plan view of the capsulorhexis apparatus shown in FIG. 2, and FIG. 4 is a view illustrating a state in which an anterior capsule incision unit shown in FIG. 2 is inserted into a guide unit. FIG. 5 is a view illustrating a capsulorhexis apparatus to which a modified anterior capsule incision unit is applied, FIG. 6 is a schematic view illustrating a capsulorhexis apparatus to which another anterior capsule incision unit is applied, and FIG. 7 is a set of diagrams illustrating a process of incising an anterior capsule with a capsulorhexis apparatus according to the present invention.

Referring to the drawings, a capsulorhexis apparatus 100 according to the present invention is intended to be inserted into an incision site of a cornea 20 or an incision site of a sclera 10 and incise a part of an anterior capsule 50 covering a crystalline lens 30. The capsulorhexis apparatus 100 includes an anterior capsule incision unit 110, a body unit 120, and a button unit 130.

The anterior capsule incision unit 110 has a closed-curve shape and is inserted into an incision site 22 of the cornea or an incision (not shown) of the sclera 10 to incise the anterior capsule 50 in a circular shape. The anterior capsule incision unit 110 heats quickly moisture of the anterior capsule 50 which comes into contact with the anterior capsule incision unit 110 using heat generated by a current applied from a current supply member (not shown) which will be described below, thereby incising the anterior capsule 50 in a circular shape.

Referring to FIG. 3, the anterior capsule incision unit 110 includes a circular incision tool 111, a moving member 115, and a current supply member (not shown), and the circular incision tool 111 may include a first wire 112 and a second wire 113 and may further include a coated wire 114.

The circular incision tool 111 functions to incise the anterior capsule 50 in a circular shape, and the moving member 115 has an end coupled to the circular incision tool 111 and functions to slidingly move the circular incision tool 111 by manipulation of the button unit 130 which will be described below.

The first wire 112 is an active electrode, and the second wire 113 is a return electrode. When a current is applied to the current supply member (not shown) through the first wire 112 which is the active electrode, the current applied through the first wire 112 flows to the second wire 113 which is the return electrode.

Unlike this embodiment, when the circular incision tool is only formed of a circular wire which is an active electrode and a counter electrode (not shown) is disposed to be spaced apart from the wire on a thigh or arm of a patient, a current applied through the wire is applied to the counter electrode (not shown) through the patient's body. Accordingly, in this case electric power varies due to individual patient's different body resistances. On the contrary, when the circular incision tool 111 according to this embodiment is formed by interposing the coated wire 114 between the first wire 112 and the second wire 113, the coated wire 114 maintains a constant resistance between the two electrodes, thereby reducing a variation in power.

An insulating coating layer (not shown) applied on a circumferential surface of the coated wire 114 may be a color contrasting with the color of an eyeball. This is intended to enable an operator to easily determine whether the circular incision tool 111 inserted into the incision site 22 of the cornea 20 is accurately positioned at the center of the front surface of the anterior capsule 50.

Since the first wire 112, the second wire 113, and the coated wire 114 are interwoven in a rope shape, elasticity of the circular incision tool 111 is increased. The first wire 112 and the second wire 113 may be a metal material having elasticity.

Referring to FIG. 5, the anterior capsule incision unit 110 is slidingly moved in the body unit 120. The anterior capsule incision unit 110 is exposed to the outside of the body unit 120 in order to incise the anterior capsule 50. When the anterior capsule 50 is not incised, the anterior capsule incision unit 110 is inserted into the body unit 120. The anterior capsule incision unit 110 may further include a current guide member 117 and a first button unit 118.

The current guide member 117 is slidingly moved and inserted into the body unit 120 or exposed to the outside of the body unit 120. When exposed to the outside thereof, the current guide member 117 is positioned at the center of the circular incision tool 111 and functions to guide a current flow to the center of the circular incision tool 111.

The current guide member 117 includes a moving bar 117a and a current guide piece 117b. The current guide member 117 is slidingly moved by manipulation of the first button unit 118 which is formed to be spaced apart from the button unit 130 on a surface of the body unit 120 and inserted into the body unit 120 or exposed to the outside of the body unit 120.

One end of the moving bar 117a is coupled to the first button unit 118 so that the other end of the moving bar 117a is moved to the center of the circular incision tool 111 by manipulation of the first button unit 118. The current guide piece 117b is integrally provided at the other end of the moving bar 117a. The current guide piece 117b functions to guide a current flowing in the circular incision tool 111 which is an active electrode to a return electrode. The current guide piece 117b guides the current flowing in the circular incision tool 111 to the inside of the circular incision tool 111 so that the anterior capsule 50 is easily incised in a circular shape.

FIG. 6 shows a modified anterior capsule incision unit 111. The modified anterior capsule incision unit 111 may include a first semicircular incision tool 111a, second semicircular incision tool 111b, a first moving member 111c, a second moving member 111d, and a current supply member 111e, and the first semicircular incision tool 111a and the second semicircular incision tool 111b may be coupled by an insulating member 111f.

The first semicircular incision tool 111a functions to incise the anterior capsule 50 in a semicircular shape, and the second semicircular incision tool 111b has a shape corresponding to the first semicircular incision tool 111a and functions to incise the anterior capsule in a semicircular shape corresponding to the semicircular shape incised by the first semicircular incision tool 111a. A plurality of wires are interwoven in a rope shape as the first semicircular incision tool 111a and the second semicircular incision tool 111b so that elasticity of the first and second semicircular incision tools 111a and 111b is increased.

The first semicircular incision tool 111a may be coupled to the first moving member 111c, and the second semicircular incision tool 111b may be coupled to the second moving member 111d. An end of the first moving member 111c is coupled to the first semicircular incision tool 111a, and the other end thereof is coupled to the button unit 130. The first moving member 111c functions to slidingly move the first semicircular incision tool 111a by manipulation of the button unit 130. An end of the second moving member 111d is coupled to the second semicircular incision tool 111b, and the other end thereof is coupled to the button unit 130. The second moving member 111d functions to slidingly move the second semicircular incision tool 111b by manipulation of the button unit 130.

The first semicircular incision tool 111a is an active electrode, and the second semicircular incision tool 111b is a return electrode. A current applied from a current supply member 111e to the first semicircular incision tool 111a flows to the second semicircular incision tool 111b, and the first semicircular incision tool 111a and the second semicircular incision tool 111b are coupled and insulated by an insulating member 111f.

The body unit 120 includes a guide unit 122 and a body 124. In the guide unit 122, the anterior capsule incision unit 110 slidingly moves, and the guide unit 122 functions to guide the anterior capsule incision unit 110 through the incision site 22 of the cornea 20.

The guide unit 122 has a silicone material, and a moving hole 122a in which the anterior capsule incision unit 110 slidingly moves may be formed in the center of the guide unit. Since the moving hole 122a is formed in the guide unit 122, the anterior capsule incision unit 110 slidingly moves in the moving hole 122a and is exposed from the inside of the moving hole 122a to the outside in the case of incising the anterior capsule 50 or is kept inserted in the moving hole 122a in the case of not incising the anterior capsule 50. Although it has been described that the guide unit 122 has a silicone material, the guide unit 122 is not limited to a silicone material and may have a material other than a silicone material.

An end of the guide unit 122 is coupled to the body 124, and the moving member 115 of the anterior capsule incision unit 110 slidingly moves in the body 124. In the body 124, a void (not shown) in which the moving member 115 slidingly moves may be formed.

The guide unit 122 coupled to the body 124 may be obliquely coupled to the body 124, and an end of the moving member 115 coupled to the circular incision tool 111 may be formed obliquely.

Since the guide unit 122 is obliquely coupled to the body 124 and the moving member 115 is obliquely formed, an operator may easily conduct an operation of inserting the guide unit 122 into the incision site 22 of the cornea 20 or an incision site (not shown) of the sclera 10 and then incising the anterior capsule 50 in a circular shape by using the circular incision tool 111.

The button unit 130 for slidingly moving the anterior capsule incision unit 110 in the body unit 120 is formed on a surface of the body unit 120. When the operator manipulates the button unit 130, the anterior capsule incision unit 110 which slidingly moves by manipulation of the button unit 130 is exposed from the inside of the body unit 120 to incise the anterior capsule 50 in a circular shape or inserted into the body unit 120.

Referring to FIG. 7, a process of incising the anterior capsule 50 by using the capsulorhexis apparatus 100 according to the present invention is schematically described as follows.

First, an operator makes the incision site 22 in the cornea 20 or the sclera 10 using an incision tool and then inserts an end of the guide unit 122 into the incision site. When the operator manipulates the button unit 130 after inserting the guide unit 122, the anterior capsule incision unit 110 is exposed to the outside of the body unit 120 and positioned on the anterior capsule 50 to be incised.

When the anterior capsule incision unit 110 positioned on the anterior capsule 50 is heated by a current, the circular incision tool 111 of the anterior capsule incision unit 110 rapidly incises a part of the anterior capsule 50. When the operator manipulates the button unit 130 in a reverse direction after incising the anterior capsule 50 in a circular shape, the anterior capsule incision unit 110 is inserted into the body unit 120 so that the anterior capsule 50, which will be incised in a circular shape by the operator, is rapidly and precisely incised. As necessary, the first button unit 118 is manipulated to use the current guide member 117 so that the current flowing in the circular incision tool 111 is guided to the center of the circular incision tool 111. Then, the anterior capsule 50 is easily incised in a circular shape.

Therefore, it is possible to precisely incise the front surface of the anterior capsule 50 covering the crystalline lens 30 in a circular shape. Consequently, revision surgery as a result of a failed surgery can be prevented and the surgical procedure can be safely and conveniently conducted. Also, the circular incision tool 111 of the anterior capsule incision unit 110 for incising the anterior capsule 50 is formed by interweaving the first wire 112 and the second wire 113 so that a current flows not through a human body but only through the circular incision tool 111. Consequently, it is possible to make an incision with stable power. Further, since the circular incision tool 111 is formed by interweaving the first wire 112 and the second wire 113, the circular incision tool 111 partially comes into contact with the anterior capsule 50. Consequently, it is possible to partially incise the anterior capsule 50 in a circular shape.

Although the present invention has been described with reference to the embodiments shown in the drawings, these embodiments are only exemplary, and those of ordinary skill in the art should appreciate that various modifications and other equivalents are possible from the embodiments. Therefore, the technical scope of the present invention should be defined by the technical scope of the claims.

### INDUSTRIAL APPLICABILITY

The present invention can be used in a capsulorhexis apparatus.

1. A capsulorhexis apparatus which is inserted into an incision site of a cornea and incises an anterior capsule covering a crystalline lens, the capsulorhexis apparatus comprising:
an anterior capsule incision unit having a closed curve shape and configured to be inserted into an incision site of an incised cornea and incise a part of an anterior capsule positioned below the cornea and generate heat by applying a current in order to incise the anterior capsule;
a body unit in which the anterior capsule incision unit slidingly moves so that the anterior capsule incision unit is exposed to an outside of the body unit when incising a part of the anterior capsule and so that the anterior capsule incision unit is inserted into the body unit when not incising the anterior capsule; and
a button unit formed on a surface of the body unit and configured to slidingly move the anterior capsule incision unit in the body unit.

2. The capsulorhexis apparatus of claim 1, wherein the anterior capsule incision unit comprises:
a circular incision tool configured to incise the part of the anterior capsule in a circular shape;
a moving member having one end coupled to the circular incision tool and another end coupled to the button unit and configured to slidingly move the circular incision tool by manipulation of the button unit; and
a current supply means configured to apply the current to the circular incision tool.

3. The capsulorhexis apparatus of claim 2, wherein the circular incision tool comprises:
a first wire which has elasticity and through which the current applied from the current supply means flows; and
a second wire which has elasticity and through which the current applied from the first wire flows.

4. The capsulorhexis apparatus of claim 3, wherein the circular incision tool further comprises a coated wire provided between the first wire and the second wire and having a circumferential surface coated with an insulating coating layer.

5. The capsulorhexis apparatus of claim 3, wherein the first wire and the second wire are interwoven in a rope shape and have a metal material.

6. The capsulorhexis apparatus of claim 2, wherein the anterior capsule incision unit further comprises a current guide member configured to slidingly move and be inserted into the body unit or exposed to the outside of the body unit and configured to be positioned at a center of the circular incision tool and guide a current flow to the center of the circular incision tool when exposed to the outside of the body unit.

7. The capsulorhexis apparatus of claim 6, wherein the circular incision tool further comprises a first button unit formed to be spaced apart from the button unit on the surface of the body unit and configured to slidingly move the current guide member to be inserted into the body unit or exposed to the outside thereof.

8. The capsulorhexis apparatus of claim 7, wherein the current guide member comprises:
a moving bar having an end coupled to the first button unit and configured to move another end to the center of the circular incision tool by manipulation of the first button unit; and
a current guide piece provided at the other end of the moving bar and configured to guide a current flow in the circular incision tool,
wherein the current guide piece is integrally provided at the other end of the moving bar.

9. The capsulorhexis apparatus of claim 1, wherein the body unit comprises:
a guide unit configured to cause one end of the anterior capsule incision unit to be inserted into the incision site of the cornea while slidingly moving in the guide unit and configured to guide the anterior capsule incision unit through the incision site of the cornea; and
a body having an end coupled to the guide unit and in which a moving member slidingly moves,
wherein the button unit is provided on a surface of the body.

10. The capsulorhexis apparatus of claim 9, wherein the guide unit has a silicone material, and
a moving hole, in which the anterior capsule incision unit slidingly moves, is formed at a center of the guide unit.

11. The capsulorhexis apparatus of claim 9, wherein an end of the guide unit coupled to the body is obliquely coupled to the body.

12. The capsulorhexis apparatus of claim 2, wherein the anterior capsule incision unit comprises:
a first semicircular incision tool configured to incise the anterior capsule in a semicircular shape;
a second semicircular incision tool having a shape corresponding to the first semicircular incision tool and configured to incise the anterior capsule in a semicircular shape;
a first moving member having an end coupled to the first semicircular incision tool and another end coupled to the button unit and configured to slidingly move the first semicircular incision tool by manipulation of the button unit;
a second moving member having an end coupled to the second semicircular incision tool and another end coupled to the button unit and configured to slidingly move the second semicircular incision tool by manipulation of the button unit; and
a current supply member configured to apply the current to the first semicircular incision tool and the second semicircular incision tool.

13. The capsulorhexis apparatus of claim 12, wherein the current is applied from the current supply member to the first semicircular incision tool, and
the current is applied from the first semicircular incision tool to the second semicircular incision tool.
